(19) United States
(12) Patent Application Publication
(10) Pub. No.: US 2025/0195777 A1
(43) Pub. Date: Jun. 19, 2025

(54) MEDICAL SYRINGE

(71) Applicant: Boston Scientific Scimed, Inc., Maple Grove, MN (US)

(72) Inventors: Michael Dotsey, Chester Springs, PA (US); Andrew Miller, Phoenixville, PA (US); Jayson Delos Santos, Alamo, CA (US); Michael Turovskiy, San Leandro, CA (US)

(73) Assignee: Boston Scientific Scimed, Inc., Maple Grove, MN (US)

(21) Appl. No.: 18/980,317

(22) Filed: Dec. 13, 2024

Related U.S. Application Data
(60) Provisional application No. 63/610,287, filed on Dec. 14, 2023.

(51) Int. Cl. A61M 5/31; A61M 5/315

(52) U.S. Cl. CPC A61M 5/3129; A61M 5/31578; A61M 2005/3142

(57) ABSTRACT

Example medical device and methods for using medical devices are disclosed. An example syringe includes a barrel having a distal end region, a proximal end region and an inner surface defining a lumen extending therein. Further, the syringe includes a plunger having a proximal end region, a distal end region and a channel, wherein the channel includes a proximal end region and a distal end region. Further, the syringe includes an engagement member coupled to the barrel. Further, the plunger is configured to be positioned within the lumen of the barrel and a portion of the engagement member extends into the channel of the plunger when the plunger is positioned within the lumen of the barrel.

### CROSS-REFERENCE TO RELATED APPLICATIONS

This application claims the benefit of priority under 35 U.S.C. § 119 of U.S. Provisional Application No. 63/610,287, filed Dec. 14, 2023, the entire disclosure of which is hereby incorporated by reference.

### TECHNICAL FIELD

The present disclosure pertains to medical devices, and methods for manufacturing medical devices. More particularly, the present disclosure pertains to syringes connected with other structures, and methods for manufacturing and using such devices.

### BACKGROUND

A wide variety of intracorporeal medical devices have been developed for medical use, for example, intravascular use. Some of these devices include syringes, aspiration devices, thrombectomy devices, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example syringe includes a barrel having a distal end region, a proximal end region and an inner surface defining a lumen extending therein. Further, the syringe includes a plunger having a proximal end region, a distal end region and a channel, wherein the channel includes a proximal end region and a distal end region. Further, the syringe includes an engagement member coupled to the barrel. Further, the plunger is configured to be positioned within the lumen of the barrel and a portion of the engagement member extends into the channel of the plunger when the plunger is positioned within the lumen of the barrel.

Alternatively or additionally to any of the embodiments above, wherein the distal end region of the barrel is configured to couple to a manifold of a catheter system.

Alternatively or additionally to any of the embodiments above, wherein the channel extends along an outer surface of the plunger.

Alternatively or additionally to any of the embodiments above, wherein the proximal end region of the channel is positioned along the proximal end region of the plunger, wherein the distal end region of the channel is positioned along the distal end region of the plunger, and wherein the channel extends from the proximal end region of the plunger to the distal end region of the plunger.

Alternatively or additionally to any of the embodiments above, wherein a portion of the channel extending between the proximal end region of the plunger and the distal end region of the plunger is substantially parallel to the longitudinal axis of the plunger.

Alternatively or additionally to any of the embodiments above, wherein the distal end region of the channel includes a curved shape.

Alternatively or additionally to any of the embodiments above, wherein engagement of the engagement member within the curved shape of the channel is configured to prevent translation and rotation of the plunger within the barrel.

Alternatively or additionally to any of the embodiments above, wherein the engagement member is positioned along the proximal end region of the barrel.

Alternatively or additionally to any of the embodiments above, wherein the engagement member includes a guide pin, and wherein a portion of the guide pin extends through a wall of the barrel.

Alternatively or additionally to any of the embodiments above, wherein the engagement member includes a spring.

Alternatively or additionally to any of the embodiments above, wherein the syringe further comprises an actuation member configured to translate relative to the plunger, and wherein translation of the actuation member disengages the spring from the channel.

Alternatively or additionally to any of the embodiments above, wherein the proximal end region of the barrel further includes a proximal flange, and wherein the distal end region of the barrel includes a distal flange.

Alternatively or additionally to any of the embodiments above, wherein the channel includes a first portion extending along a longitudinal axis of the plunger and a second portion extending at an angle relative to the first portion.

Alternatively or additionally to any of the embodiments above, wherein the distal end region of the plunger further includes a stopper, and where the stopper is configured to seal against the inner surface of the barrel.

Another example syringe includes a barrel having a distal end region, a proximal end region and an inner surface defining a lumen extending therein. Further, the syringe includes a plunger having a proximal end region, a distal end region, a handle and a first detent. The syringe further includes an actuation member coupled to the plunger and a spring coupled to the barrel, wherein the spring includes an engagement portion configured to engage the first detent. Further, the plunger is configured to be positioned within the lumen of the barrel and the engagement of the engagement portion of the spring within the first detent prevents translation of the plunger within the barrel.

Alternatively or additionally to any of the embodiments above, further comprising a second detent, and wherein the first detent is spaced away from the second detent.

Alternatively or additionally to any of the embodiments above, wherein the plunger further includes a channel, and wherein the actuation member is disposed within at least a portion of the channel.

Alternatively or additionally to any of the embodiments above, wherein the actuation member is configured to translate relative to the plunger, and wherein translation of the actuation member disengages the spring from the first detent or the second detent.

Alternatively or additionally to any of the embodiments above, wherein a proximal end of the actuation member extends through an aperture of the handle.

Another example syringe includes a barrel having a distal end region, a proximal end region and an inner surface defining a lumen extending therein. The syringe further includes a plunger having an outer surface and a plurality of teeth extending along the outer surface. The syringe further includes an actuation lever coupled to the barrel. Further, the actuation lever includes a projection configured to engage a first tooth and a second tooth of the plurality of teeth the plunger is configured to be positioned within the lumen of the barrel and engagement of the projection with the plurality of teeth prevents the plunger from translating distally relative to the barrel.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an example syringe including a plunger positioned within a lumen of a barrel;

FIG. 2 illustrates the syringe of FIG. 1 in a first configuration;

FIG. 3 illustrates the syringe of FIG. 1 in a second configuration;

FIG. 4 is a perspective view of another example syringe including a plunger positioned within a lumen of a barrel;

FIG. 5 is a perspective view of another example syringe including a plunger positioned within a lumen of a barrel;

FIG. 6 illustrates the syringe of FIG. 5 in a first configuration;

FIG. 7 illustrates the syringe of FIG. 5 in a second configuration;

FIG. 8 illustrates the syringe of FIG. 5 in a third configuration;

FIG. 9A is a perspective view of another example syringe including a plunger positioned within a lumen of a barrel;

FIG. 9B is a perspective view of a portion of the syringe shown in FIG. 9A;

FIG. 9C is a perspective view of a portion of the syringe shown in FIG. 9A;

FIG. 10 is a perspective view of another example syringe including a plunger positioned within a lumen of a barrel;

FIG. 11 illustrates the syringe of FIG. 10 in a first configuration;

FIG. 12 illustrates the syringe of FIG. 10 in a second configuration;

FIG. 13 is a perspective view of another example syringe including a plunger positioned within a lumen of a barrel;

FIG. 14 illustrates the syringe of FIG. 13 in a first configuration;

FIG. 15 illustrates the syringe of FIG. 13 in a second configuration; and

FIG. 16 illustrates the syringe of FIG. 13 in a third configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Thromboembolism may be characterized as an adverse medical condition whereby a blood clot forms an occlusion in a blood vessel. In some instances, a blood clot (thrombus) having formed in a blood vessel, dislodges and blocks another blood vessel. Blood clots in both the arterial system and the venous system may cause serious harm. For example, when an artery is occluded by a thrombus, tissue ischemia may develop. The ischemia may eventually progress to tissue infarction if the occlusion persists. Similarly, in the venous system, thrombus may also cause serious harm. For example, thrombus may develop in the veins of the legs, a condition known as deep venous thrombosis (DVT). DVT can obstruct the flow of venous blood from the legs, thereby leading to adverse conditions such as swelling and infection. Additionally, in some instances a blood clot in the legs may break loose, travel through the bloodstream to the lungs and cause a sudden blockage in a lung artery, a condition known as a pulmonary embolism. Pulmonary embolism can lead to permanent damage to the lungs, low oxygen levels in the blood and damage to other organs due to the lack of oxygen.

However, it can be appreciated that the re-establishment of sufficient blood flow may significantly reduce harm caused by thromboembolism. A variety of medical treatments exist to re-establish blood flow through an occluded vessel. For example, in some instance medicines which include anticoagulants may be utilized to prevent the blood clots from forming while thrombolytics may be utilized to dissolve existing blood clots. However, not all patients can be treated with anticoagulants and/or thrombolytics. Therefore, in some instances, medical devices may be utilized to mechanically extract (e.g., aspirate) a volume of thrombus via a catheter-based intervention. For example, a catheter coupled to an aspiration device (e.g., syringe) may be utilized to aspirate thrombus. Accordingly, it can be appreciated that the syringe may be designed to create maximum vacuum power sufficient to dislodge and extract the thrombus through the catheter lumen. Aspiration devices designed to maximize vacuum power through an aspiration catheter are disclosed herein.

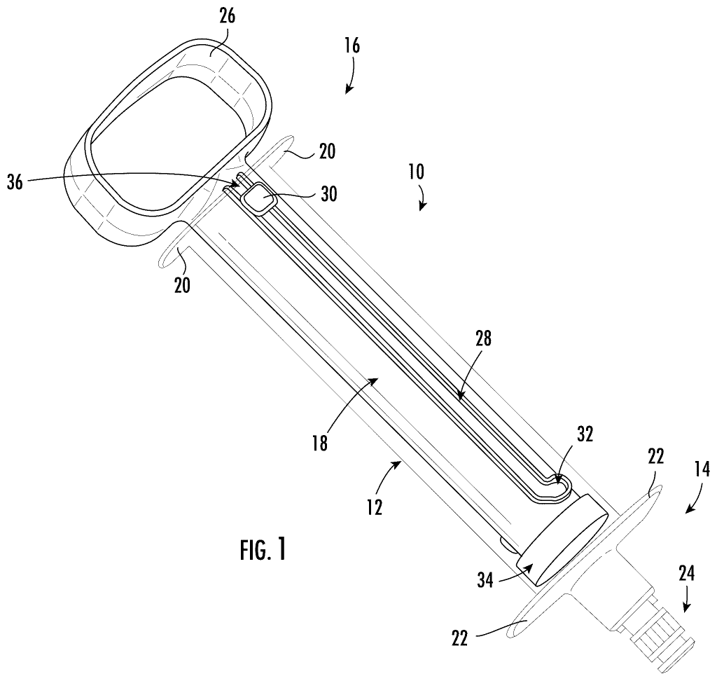

FIG. 1 is a side view of an example aspiration source 10 (e.g., syringe). It can be appreciated that the syringe 10 may include a barrel 12 having a lumen (e.g., inner chamber, cavity, etc.) within which a plunger 18 (e.g., piston) may be positioned. The barrel 12 of the syringe 10 may include a distal end region 14 and a proximal end region 16. In some instances, the proximal end region 16 of the barrel 12 may include one or more proximal finger flanges 20 (e.g., ergonomic finger grips) extending radially away from the outer surface of barrel 12. Additionally, the distal end region 14 of the barrel 12 may include one or more distal flanges 22 (e.g., ergonomic finger grips) extending radially away from the outer surface of barrel 12. Further, the proximal end of the plunger 18 may include a plunger handle 26 designed to permit a clinician to advance and/or retract the plunger 18 within the lumen of the barrel 12. It can be appreciated that a clinician may utilize the one or more proximal finger flanges 20 and or the distal flanges 22 in conjunction with the plunger handle 26 to advance and/or retract the plunger 18 within the lumen of the barrel 12. The distal flanges 22 may be gripped or wrapped by multiple fingers, a hand or first to provide additional support when advancing or retracting the plunger handle 26.

FIG. 1 illustrates the syringe 10 having the plunger 18 slidably and rotatably positioned within a lumen of the barrel 12. In some examples, the cross-sectional shape of the plunger 18 may be substantially cylindrical. However, in other examples, the cross-sectional shape of the plunger 18 may include other shapes, including ovular, triangular, square, rectangular, star-shaped, polygonal or other similar shapes. Further, in some examples, it can be appreciated that the plunger 18, the barrel 12 or both the plunger 18 and the barrel 12 may be constructed from a transparent material which permits visualization of contents within the lumen of the barrel 12. For example, the plunger 18, the barrel 12 or both the plunger 18 and the barrel 12 may be constructed from a polymeric, transparent material which may permit the visualization of a blood clot after being aspirated into the lumen of the barrel 12. An exemplary material for the plunger 18, the barrel 12, or both the plunger 18 and the barrel 12 is polycarbonate.

Additionally, FIG. 1 illustrates that the distal end region 14 of the barrel 12 may include one or more features designed to couple the barrel 12 of the syringe 10 to another medical device (e.g., an aspiration catheter). It can be appreciated that the distal end region 14 of the barrel 12 may include a coupling element 24 which permits the distal end region 14 of the barrel 12 to be rotated (e.g., screwed) and attached to a manifold of an aspiration catheter. For example, it can be appreciated that the coupling element 24 positioned along the distal end region 14 of the barrel 12 may include a standard luer fitting (e.g., luer lock) that can be coupled to a manifold of an aspiration catheter.

FIG. 1 further illustrates that the plunger 18 may include a stopper (e.g., gasket, etc.) 34 positioned along the distal end of the plunger 18. In some examples, the stopper 34 may include an O-ring which extends circumferentially around the outer surface of the distal end of the plunger 18. For example, the stopper 34 may include an O-ring which is nested in a circumferential groove formed in the outer surface of the plunger 18. It can be appreciated that the stopper 34 of the syringe 10 may be designed to seal against an inner surface of the of the barrel 12. Additionally, it can be further appreciated that the seal provided by the stopper 34 against the inner surface of the barrel 12 may permit the plunger 18 to generate a vacuum force when retracted in a distal-to-proximal direction relative to the barrel 12. Vacuum force is dependent upon several factors including the overall volume of the barrel 12.

FIG. 1 further illustrates that the plunger 18 may further include a channel 28 (e.g., motion channel, track, guide, groove, etc.) extending along the longitudinal length of the plunger 18. For example, FIG. 1 illustrates that the channel 28 may include a first end 36 positioned adjacent to the plunger handle 26 and a second end 32 positioned adjacent to the stopper 34, whereby the channel 28 extends longitudinally along the outer surface of the plunger 18 from the first end 36 to the second end 32. Additionally, FIG. 1 illustrates that the second end 32 of the channel may include a curved shape (e.g., “J-shape”), whereby the channel 28 transitions from extending along the longitudinal axis of the plunger 18 to extending along the circumference of the outer surface of the plunger 18. In other words, the channel 28 illustrated in FIG. 1 may extend along the outer surface of the plunger 18 from the first end 36 toward the second end 32, whereby the channel 28 bends approximately 90 degrees and extends along the outer circumference of the plunger 18.

FIG. 1 further illustrates that the syringe 10 may further include a motion guide pin 30. It can be appreciated that at least a portion of the pin 30 may be disposed along the outer surface of the barrel 12 and that another portion of the pin 30 may extend through the wall of the barrel 12 and engage (e.g., nest within, project into, extend into, etc.) the channel 28. In other words, the pin 30 may include an attachment region which is attached to the outer surface of the barrel 12 and projection portion which extends through the wall of the barrel 12 and engages and/or projects into the channel 28.

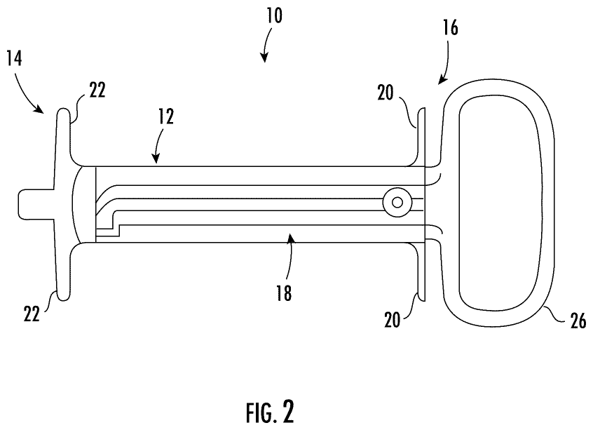

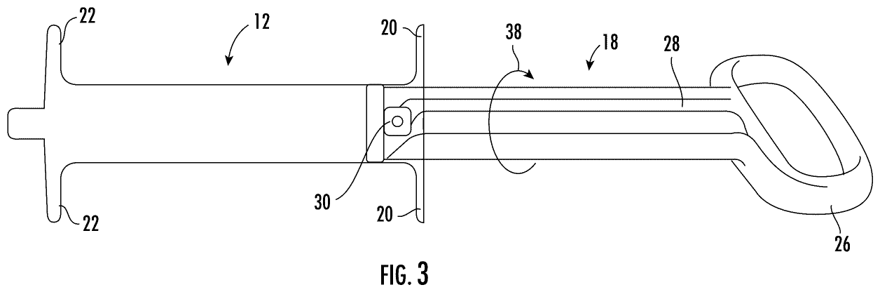

FIGS. 2-3 illustrate the proximal retraction of the plunger 18 relative to the barrel 12 of the syringe 10. FIG. 2 illustrates the plunger 18 fully advanced in a distal direction within the barrel 12 whereby the plunger 18 has filled the entire space of the lumen of the barrel 18. In this configuration, a distally-facing surface of the stopper 34 may contact a proximal facing inner surface of the barrel 12. Further, it can be appreciated from FIG. 2 that the plunger handle 26 is positioned such that it is adjacent to the proximal finger flanges 20. It can be further appreciated that FIG. 2 may represent a pre-retraction configuration in which a clinician may position the plunger 18 to generate a maximum aspiration force (e.g., a maximum suction force) because the plunger 18 has extended a maximum distance into the barrel 12. It can be appreciated that a clinician may position the plunger 18 in a different pre-retraction configuration whereby the plunger 18 is partially inserted into the barrel 12 (e.g., a configuration whereby the stopper 34 is positioned at a location midway between the distal end region 14 and the proximal end region 16 of the barrel 12) to generate an aspiration force which is less than a maximum aspiration force.

FIG. 3 illustrates that the syringe 10 shown in FIG. 2 after the plunger 18 has been proximally retracted such that the stopper 34 has translated along an inner surface of the barrel 12 from the distal end region 14 to the proximal end region 16 of the barrel 12. It can be appreciated that to retract the plunger 18, a clinician may utilize the one or more of the proximal finger flanges 20 and or the distal flanges 22 in conjunction with the plunger handle 26 to retract the plunger 18 within the lumen of the barrel 12.

Further, FIG. 3 illustrates that during the retraction of the plunger 18, the engagement of the pin 30 within the channel 28 forces the plunger 18 to be retracted along a longitudinal retraction path which mirrors the orientation of the channel 28 relative to the barrel 12. In other words, because the pin 30 is fixedly attached to the barrel 12, the pin 30 will maintain the plunger 18 on a retraction path defined by the orientation and shape of the channel 28 relative to the barrel 12. Accordingly, FIG. 3 illustrates that the plunger 18 may be retracted along a retraction pathway which is void of any rotation relative to the barrel 12 up until the second end 32 of the channel 28 reaches the pin 30, whereby continued engagement of the pin 30 within the channel 28 directs the plunger 18 to rotate (depicted by the arrow 38) in a clockwise direction (as viewed from and end view of the handle 26). It can be appreciated that engagement of the pin 30 within the second end 32 of the channel 28 may maintain the plunger 18 in a retracted position whereby the plunger 18 will not distally advance due to the negative pressure being imparted on the plunger 18 via the retraction of the plunger 18. It can be further appreciated that to release the plunger 18 and permit it to distally advance, a clinician may initiate rotation of the plunger 18 in a counter-clockwise direction (as viewed from an end view of the handle 26) thereby allowing the plunger 18 to advance along the portion of the channel 28 which is aligned with the longitudinal axis of the barrel 12.

Additionally, it can be appreciated that the syringe 10 (or any of the syringes disclosed herein) may be attached to an aspiration catheter. Further, the aspiration catheter may include a clamp, valve, or stopcock which may be utilized to control flow through the catheter. It can be further appreciated that, at the clinician's discretion, the clamp, valve, or stopcock may be opened whereby a vacuum pressure generated by the syringe 10 may create a vacuum force (e.g., suction) through the aspiration catheter which is attached to the syringe 10. It can be appreciated that the vacuum force generated through the aspiration catheter may aspirate blood clots or thrombus material located adjacent the distal end aspiration catheter.

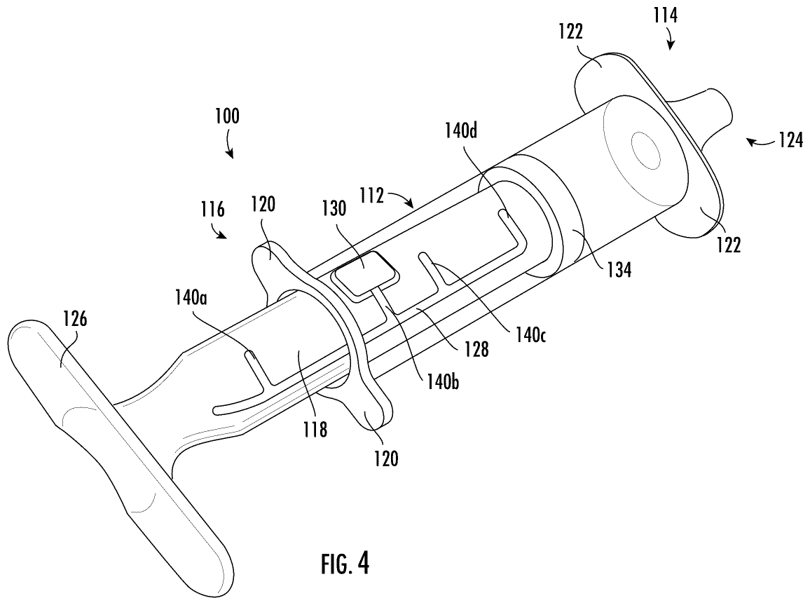

FIG. 4 is a perspective view of another example syringe 100. The syringe 100 may be similar in form and function to the syringe 10 described herein. For example, the syringe 100 may include a barrel 112 having a lumen (e.g., inner chamber, cavity, etc.) within which a plunger 118 (e.g., piston) may be positioned. The barrel 112 of the syringe 100 may include a distal end region 114 and a proximal end region 116. In some instances, the proximal end region 116 of the barrel 112 may include one or more proximal finger flanges 120 (e.g., ergonomic finger grips) extending radially away from the outer surface of barrel 112. Additionally, the distal end region 114 of the barrel 112 may include one or more distal flanges 122 (e.g., ergonomic grips) extending radially away from the outer surface of barrel 112. It can be appreciated that a clinician may utilize the one or more proximal finger flanges 120 and or the distal flanges 122 in conjunction with the plunger handle 126 to advance and/or retract the plunger 118 within the lumen of the barrel 112. The distal flanges 122 may be gripped or wrapped by multiple fingers, a hand or first to provide additional support when advancing or retracting the plunger handle 26.

Additionally, FIG. 4 illustrates that the distal end region 114 of the barrel 112 may include one or more features designed to couple the barrel 112 of the syringe 100 to another medical device (e.g., an aspiration catheter). It can be appreciated that the distal end region 114 of the barrel 112 may include a coupling element 124 which permits the distal end region 114 of the barrel 112 to be rotated (e.g., screwed) and attached to a manifold of an aspiration catheter. For example, it can be appreciated that the coupling element 124 positioned along the distal end region 114 of the barrel 112 may include a standard luer fitting (e.g., luer lock) that can be coupled to a manifold of an aspiration catheter.

FIG. 4 further illustrates that the plunger 118 may include a stopper (e.g., gasket, etc.) 134 positioned along the distal end of the plunger 118. In some examples, the stopper 134 may include an O-ring which extends circumferentially around the outer surface of the distal end of the plunger 118. For example, the stopper 134 may include an O-ring which is nested in a circumferential groove formed in the outer surface of the plunger 118. It can be appreciated that the stopper 134 of the syringe 100 may be designed to seal against an inner surface of the of the barrel 112. Additionally, it can be further appreciated that the seal provided by the stopper 134 against the inner surface of the barrel 112 may permit the plunger 118 to generate a vacuum force when retracted in a distal-to-proximal direction relative to the barrel 112.

FIG. 4 further illustrates that the plunger 118 may further include a channel 128 (e.g., motion channel, track, guide, groove, etc.) extending along the longitudinal length of the plunger 118. For example, FIG. 4 illustrates that the channel 128 may include a plurality of locking channels 140a, 140b, 140c, 140d which extend along the circumference of the plunger 118. It can be appreciated from FIG. 4 that each of the locking channels 140a, 140b, 140c, 140d may be oriented substantially perpendicular to the channel 128. For example, each of the locking channels 140a, 140b, 140c, 140d may extend approximately 90 degrees relative to the channel 128. Further, FIG. 4 illustrates that each of the locking channels 140a, 140b, 140c, 140d may be spaced away from one another long the longitudinal axis of the plunger 118. It can be appreciated that the relative spacing between each of the locking channels 140a, 140b, 140c, 140d may correspond to a given volume of fluid and/or vacuum generated when the plunger 118 is proximally retracted.

FIG. 4 further illustrates that the syringe 100 may further include a motion guide pin 130. It can be appreciated that at least a portion of the pin 130 may be disposed along the outer surface of the barrel 112 and that another portion of the pin 130 may extend through the wall of the barrel 112 and engage (e.g., nest within, project into, extend into, etc.) the channel 128 and/or a locking channel 140a, 140b, 140c, 140d. In other words, the pin 130 may include an attachment region which is attached to the outer surface of the barrel 112 and projection portion which extends through the wall of the barrel 112 and engages and/or projects into the channel 128 and/or a locking channel 140a, 140b, 140c, 140d.

It can be appreciated that to retract the plunger 118, a clinician may utilize the one or more of the proximal finger flanges 120 and or the distal flanges 122 in conjunction with the plunger handle 126 to retract the plunger 118 within the lumen of the barrel 112. Further, FIG. 4 illustrates that during the retraction of the plunger 118, the pin 130 may force the plunger 118 to be retracted along a longitudinal retraction path defined by the channel 128 and/or a locking channel 140a, 140b, 140c, 140d. In other words, because the pin 130 is fixedly attached to the barrel 112, the pin 130 will maintain the plunger 118 on a retraction path defined by the orientation and shape of the channel 128 and/or a locking channel 140a, 140b, 140c, 140d relative to the barrel 112. Accordingly, FIG. 4 illustrates that, if desired, the plunger 118 may be retracted along the entire length of the channel 128. In other words, a clinician may retract the plunger 118 along the entire length of the channel 128 without rotating the plunger 118 such that the pin 130 engages one of the locking channels 140a, 140b, 140c, 140d.

Additionally, it can be appreciated that during retraction of the plunger 118, a clinician may rotate the plunger 118 in a clockwise direction (as viewed from an end view of the handle 26) to engage the pin 130 within one of the locking channels 140a, 140b, 140c, 140d. For example, FIG. 4 illustrates the plunger 118 having been rotated such that the pin 130 is engaged with the locking channel 140b. It can be appreciated that engagement of the plunger 118 in a locking channel 140a, 140b, 140c, 140d may maintain the plunger 118 in a retracted position whereby the plunger 118 will not advance in a proximal-to-distal direction due to the negative pressure being imparted on the plunger 118 via the retraction of the plunger 118. It can be further appreciated that to release the plunger 118 and permit it to distally advance, a clinician may initiate rotation of the plunger 118 in a counter-clockwise direction (as viewed from an end view of the handle 126) thereby allowing the plunger 118 to advance along the channel 128 which is aligned with the longitudinal axis of the barrel 112.

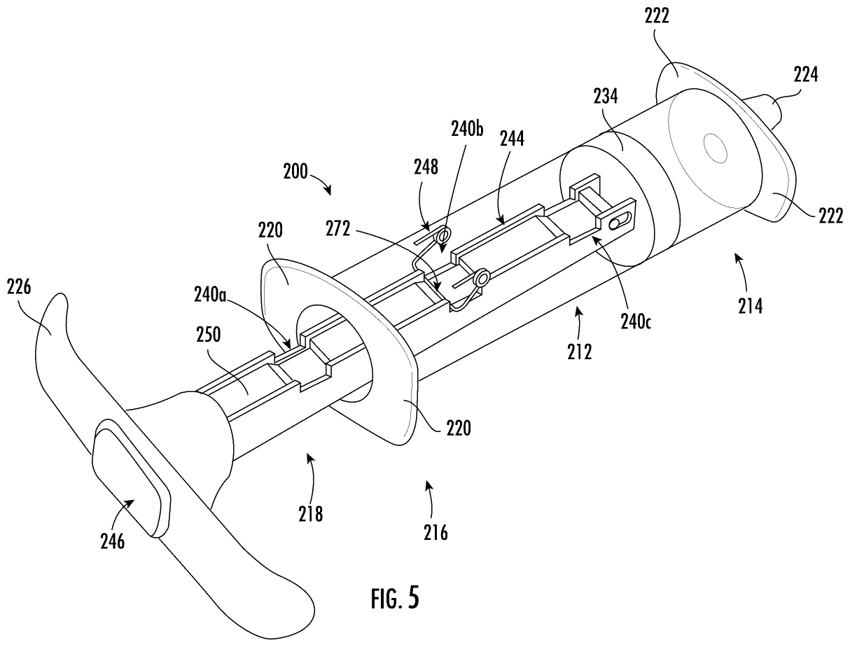

FIG. 5 is a perspective view of another example syringe 200. The syringe 200 may be similar in form and function to other syringes described herein. For example, the syringe 200 may include a barrel 212 having a lumen (e.g., inner chamber, cavity, etc.) within which a plunger 218 (e.g., piston) may be positioned. The barrel 212 of the syringe 200 may include a distal end region 214 and a proximal end region 216. In some instances, the proximal end region 216 of the barrel 212 may include one or more proximal finger flanges 220 (e.g., ergonomic finger grips) extending radially away from the outer surface of barrel 212. Additionally, the distal end region 214 of the barrel 212 may include one or more distal flanges 222 (e.g., ergonomic grips) extending radially away from the outer surface of barrel 212. It can be appreciated that a clinician may utilize the one or more proximal finger flanges 220 and/or the distal flanges 222 in conjunction with the plunger handle 226 to advance and/or retract the plunger 218 within the lumen of the barrel 212. The distal flanges 222 can be gripped or wrapped by multiple fingers, a hand or first to provide additional support when advancing or retracting the plunger handle 26.

Additionally, FIG. 5 illustrates that the distal end region 214 of the barrel 212 may include one or more features designed to couple the barrel 212 of the syringe 200 to another medical device (e.g., an aspiration catheter). It can be appreciated that the distal end region 214 of the barrel 212 may include a coupling element 224 which permits the distal end region 214 of the barrel 212 to be rotated (e.g., screwed) and attached to a manifold of an aspiration catheter. For example, it can be appreciated that the coupling element 224 positioned along the distal end region 214 of the barrel 212 may include a standard luer fitting (e.g., luer lock) that can be coupled to a manifold of an aspiration catheter.

FIG. 5 further illustrates that the plunger 218 may include a stopper (e.g., gasket, etc.) 234 positioned along the distal end of the plunger 218. In some examples, the stopper 234 may include an O-ring which extends circumferentially around the outer surface of the distal end of the plunger 218. For example, the stopper 234 may include an O-ring which is nested in a circumferential groove formed in the outer surface of the plunger 218. It can be appreciated that the stopper 234 of the syringe 200 may be designed to seal against an inner surface of the of the barrel 212. Additionally, it can be further appreciated that the seal provided by the stopper 234 against the inner surface of the barrel 212 may permit the plunger 218 to generate a vacuum force when retracted in a distal-to-proximal direction relative to the barrel 212.

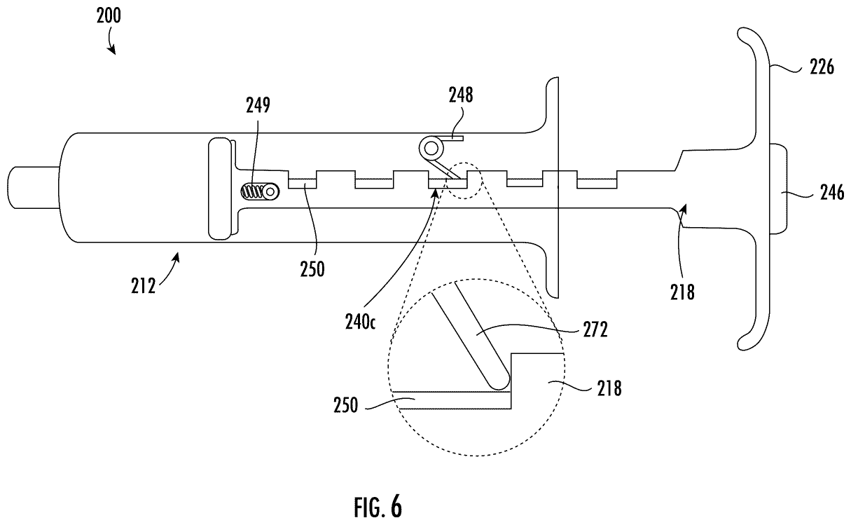

FIG. 5 further illustrates that the syringe 200 may further include an actuation member 250 nested (e.g., disposed, positioned, etc.) within a channel defined by the plunger 218. The actuation member 250 may be actuated via pushing a first end 246 (e.g., push button 246) of the actuation member 250 which extends through a portion of the handle 226 (as shown in FIG. 5). As will be described in greater detail with respect to FIGS. 6-8, pressing the first end 246 of the actuation member 250 may actuate the actuation member 250 such that the actuation member 250 shifts in a proximal-to-distal direction relative to the plunger 218.

FIG. 5 further illustrates that the plunger 218 may include one or more detents 240a, 240b, 240c. The plunger may include 1, 2, 3, 4, 5, 6 or more detents. For purposes herein, each of the detents 240a, 240b, 240c may be defined as notch or groove formed within the wall of the plunger 218. Additionally, FIG. 5 illustrates that the syringe 200 may further include a spring 248 disposed along an inner surface of the barrel 212. As illustrated in FIG. 5, the spring 248 may include one or more legs which may be attached to an inner surface of the barrel 212. Further, FIG. 5 illustrates that the spring 248 may also include an engagement portion 272 which may engage a detent 240a, 240b, 240c of the plunger 218. For example, FIG. 5 illustrates the engagement portion 272 of the spring 248 engaged with the detent 240b of the plunger 218. It can be appreciated from FIG. 5 that the engagement of the engagement portion 272 of the spring 248 within the detent 240b may prevent plunger 218 from moving in a distal direction relative to the barrel 212. The force of the spring 248 shown in FIG. 5, and in other embodiments, may be less than or equal to 22.2 Newtons (N) (5 Pounds-Force (lbf)), less than or equal to 17.8 N (4 lbf), less than or equal to 13.3 N (3 lbf), less than or equal to 8.9 N (2 lbf), less than or equal to 4.4 N (1 lbf), or greater than or equal to 2.2 N (0.5 lbf).

It can be appreciated from FIG. 5 that the orientation of the spring 248 relative to the plunger 218 may permit a clinician to utilize the one or more of the proximal finger flanges 220 and or the distal flanges 222 in conjunction with the plunger handle 226 to retract the plunger 218 within the lumen of the barrel 212. For example, it can be appreciated that as clinician pulls the handle 226 of the plunger 218 in a proximal direction, the orientation of the spring 248 permits the spring 248 to slide along the surface of the detent 240b, whereby when the spring 248 engages the distal end of the detent 240b, the spring 248 may be squeezed (e.g., pinched, flexed, etc.) to permit the engagement portion 272 of the spring 248 to compress toward an inner surface of the barrel 212 and slide along an upper surface 244 of the plunger 218 (and thereby permit the proximal retraction of the plunger 218).

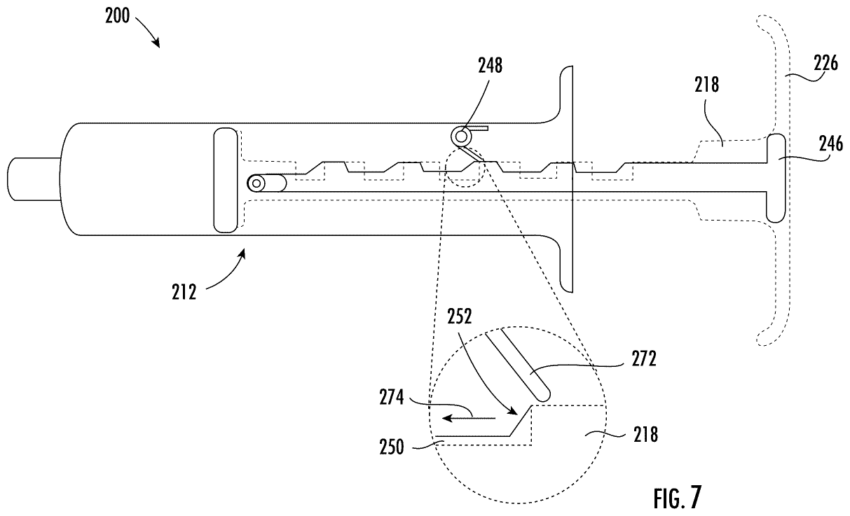

FIGS. 6-8 illustrate the actuation of the actuation member 250 of the plunger 218 which permits the plunger 218 to be advanced distally (versus the proximal retraction of the plunger 218 as described above). The detailed view of FIG. 6 illustrates engagement portion 272 of the spring 248 engaged within the detent 240c of the plunger 218. For example, the detailed view of FIG. 6 illustrates the engagement portion 272 resting on the actuation member 250 and engaged with a distally facing surface of the plunger 218. As described herein, engagement of the engagement portion 272 of the spring 248 within the detent 240c of the plunger 218 may prevent the distal advancement of the plunger 218 relative to the barrel 212. As illustrated in FIG. 6, it can be appreciated that the syringe 200 further include a spring 249 which may apply a force upon the actuation member 250, whereby the force imparted upon the actuation member 250 is directed toward the first end 246 of the actuation member 250. As will discussed with respect to FIG. 7, the spring force imparted upon the actuation member 250 directed toward the first end 246 may be overcome by depressing the first end 246 of the actuation member 250.

FIG. 7 illustrates the actuation of the actuation member 250 which compresses the spring 248 and permits the distal advancement of the plunger 218 relative to the barrel 212 (for clarity, the plunger 218 is shown in a dashed outline in FIG. 7). For example, FIG. 7 illustrates that the first end 246 of the actuation member 250 has been depressed relative to the handle 226, whereby pressing the first end 246 of the actuation member 250 overcomes the applied force of the spring 249 and advances the actuation member 250 distally relative to the plunger 218. The distal advancement of the actuation member 250 relative to the plunger is depicted by the arrow 274 shown in the detailed view of FIG. 7.

Additionally, the detailed view of FIG. 7 illustrates that the distal advancement of the actuation member 250 may advance a tapered face 252 of the actuation member 250 toward the engagement portion 272 of the spring. It can be appreciated that as the tapered face 252 of the actuation member 250 engages the engagement portion 272 of the spring 248, the tapered face 252 may drive the engagement portion 272 of the spring 248 toward an inner surface of the barrel 212 (e.g., compressing the spring 248) and permitting the engagement portion 272 of the spring 248 to engage and slide along an upper surface 244 of the plunger 218 (and thereby permitting the distal advancement of the plunger 218). In some examples, the tapered face 252 may be angled between about 30 to 45 degrees West of South when viewed in FIG. 7. The angle of the tapered face 252 may define the magnitude of the release force and the release distance that the engagement portion 272 needs to travel to release the engagement portion 272 from the detent 240c of the plunger 218. It can be appreciated that shallowing the angle of the tapered face 252 may result in less force, but a longer release distance, required to release the engagement portion 272 from the detent 240c of the plunger 218.

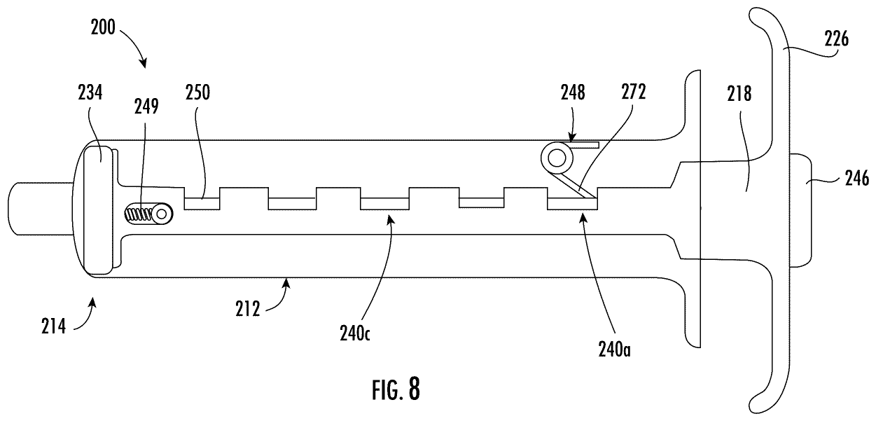

FIG. 8 illustrates the syringe 200 after the plunger 218 has been advanced distally such that the engagement portion 272 of the spring is engaged within the detent 240a of the plunger 218. FIG. 8 illustrates that the first end 246 of the actuation member 250 is not depressed and the spring 249 is applying a proximal force which pushes the first end 246 of the actuation member 250 proximally through an aperture in the handle 226. It can be appreciated that the engagement of the engagement portion 272 of the spring 248 within the detent 240a represents a configuration in which the plunger 218 has been distally advanced to a maximum extent, whereby the stopper 234 may engage the distal inner surface of the barrel 212.

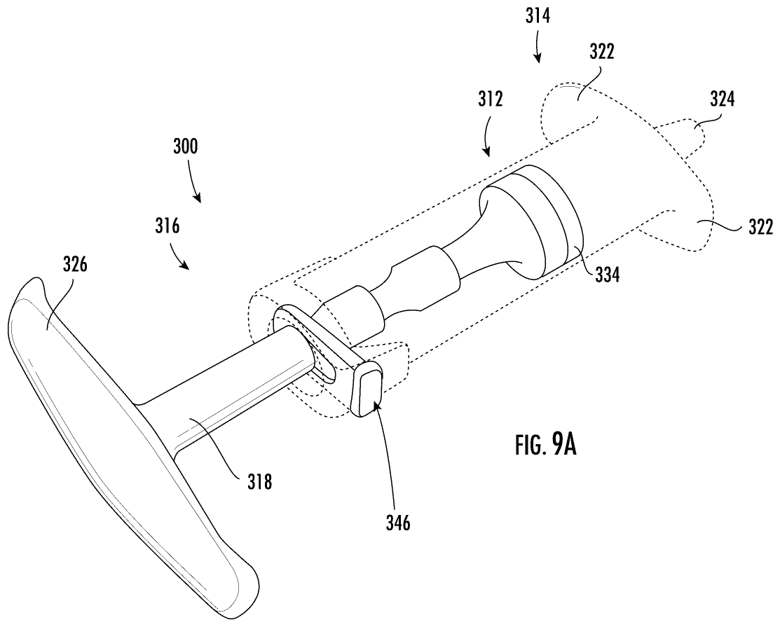

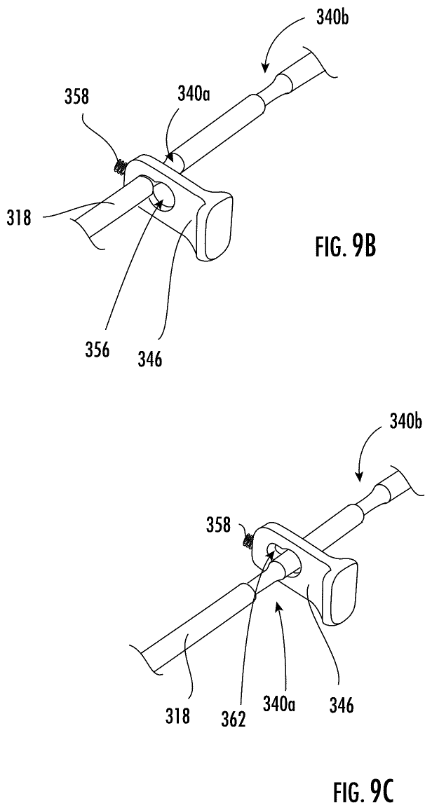

FIG. 9A is a perspective view of another example syringe 300. The syringe 300 may be similar in form and function to other syringes described herein. For example, the syringe 300 may include a barrel 312 having a lumen (e.g., inner chamber, cavity, etc.) within which a plunger 318 (e.g., piston) may be positioned (for clarity, the barrel 312 is shown in a dashed outline in FIG. 9A). The barrel 312 of the syringe 300 may include a distal end region 314 and a proximal end region 316. In some instances, the distal end region 314 of the barrel 312 may include one or more distal flanges 322 (e.g., ergonomic grips) extending radially away from the outer surface of barrel 312. It can be appreciated that a clinician may utilize the distal flanges 322 in conjunction with the plunger handle 326 to advance and/or retract the plunger 318 within the lumen of the barrel 312. The distal flanges 322 can be gripped or wrapped by multiple fingers, a hand or first to provide additional support when advancing or retracting the plunger handle 26.

Additionally, FIG. 9A illustrates that the distal end region 314 of the barrel 312 may include one or more features designed to couple the barrel 312 of the syringe 300 to another medical device (e.g., an aspiration catheter). It can be appreciated that the distal end region 314 of the barrel 312 may include a coupling element 324 which permits the distal end region 314 of the barrel 312 to be rotated (e.g., screwed) and attached to a manifold of an aspiration catheter. For example, it can be appreciated that the coupling element 324 positioned along the distal end region 314 of the barrel 312 may include a standard luer fitting (e.g., luer lock) that can be coupled to a manifold of an aspiration catheter.

FIG. 9A further illustrates that the plunger 318 may include a stopper (e.g., gasket, etc.) 334 positioned along the distal end of the plunger 318. In some examples, the stopper 334 may include an O-ring which extends circumferentially around the outer surface of the distal end of the plunger 318. For example, the stopper 334 may include an O-ring which is nested in a circumferential groove formed in the outer surface of the plunger 318. It can be appreciated that the stopper 334 of the syringe 300 may be designed to seal against an inner surface of the of the barrel 312. Additionally, it can be further appreciated that the seal provided by the stopper 334 against the inner surface of the barrel 312 may permit the plunger 318 to generate a vacuum force when retracted in a distal-to-proximal direction relative to the barrel 312.

FIG. 9A further illustrates that the syringe 300 may further include an actuation member 346 coupled to barrel 312 and the plunger 318. For example, as illustrated in FIG. 9A, the actuation member 346 may extend through a wall of the barrel 312. The actuation member 346 may actuate in a direction that is substantially perpendicular to the barrel 312. Additionally, as shown in FIGS. 9B-9C, the syringe 300 may further include a spring 358 which exerts a radially outward lateral force on the actuation member 346. The lateral force applied to the actuation member 346 by the spring 258 may be overcome by pushing (e.g., depressing, etc.) the actuation member 346 laterally inward. As will be described within respect to FIGS. 9A-9B, pressing the actuation member 346 laterally inward may permit a clinician to either retract or advance the plunger 318 relative to the barrel 312.

FIG. 9B illustrates a configuration of the syringe 300 (shown in FIG. 9A) in which the plunger 318 is extending through a first aperture 362 (shown in FIG. 9C) of the actuation member 346 (the barrel 312 has been omitted from FIGS. 9B-9C for clarity purposes). FIG. 9B may represent a configuration in which the actuation member 346 is not being pressed laterally inward. FIG. 9B further illustrates that the actuation member 346 may further include a second aperture 356 positioned adjacent to the first aperture 362 (shown in FIG. 9C). The second aperture 356 may include a diameter which is larger than the first aperture 362. For example, the diameter of the second aperture 356 may be between 0.953 centimeters (cm) (0.375 inches) and 1.27 cm (0.5 inches) while the diameter of the first aperture 362 may be between 0.635 cm (0.25 inches) and 0.953 cm (0.375 inches).

Further, FIG. 9B illustrates that the plunger 318 may include a first narrowed (e.g., necked) region 340a spaced away from a second narrowed (e.g., necked) region 340b. The first narrowed region 340a and the second narrowed region 340b may be defined as regions along the plunger 318 in which the diameter of the plunger 318 is reduced. In other words, the diameter of first narrowed region 340a and the second narrowed region 340b may be less than the diameter of the portions of the plunger 318 which are immediately adjacent to the first narrowed region 340a and the second narrowed region 340b.

FIG. 9B further illustrates that the diameter of the first aperture 362 may be sized to permit the first narrowed portion 340a to extend therethrough. However, it can be further appreciated that the first aperture 362 may be sized to not permit the portions of the plunger 318 which are immediately adjacent to the first narrowed region 340a to pass therethrough. Accordingly, it can be appreciated from FIG. 9B that when the actuation member is not being pressed laterally inward (as is the configuration of FIG. 9A and FIG. 9B), the plunger 318 is prevented from being either proximally retracted or distally advanced.

FIG. 9C illustrates a configuration of the syringe 300 (shown in FIG. 9A) in which the actuation member 346 has been pressed laterally inward (via a clinician, for example). It can be appreciated from FIG. 9C that pressing the actuation member 346 laterally inward may permit the plunger 318 to pass through the second aperture 356 (shown in FIG. 9B). Accordingly, pressing the actuation member 346 laterally inward may release the first narrowed region 340a from the first aperture 362, thereby allowing the plunger 318 to freely pass through the first aperture 356 and permitting a clinician to either retract or advance the plunger 318 relative to the barrel 312.

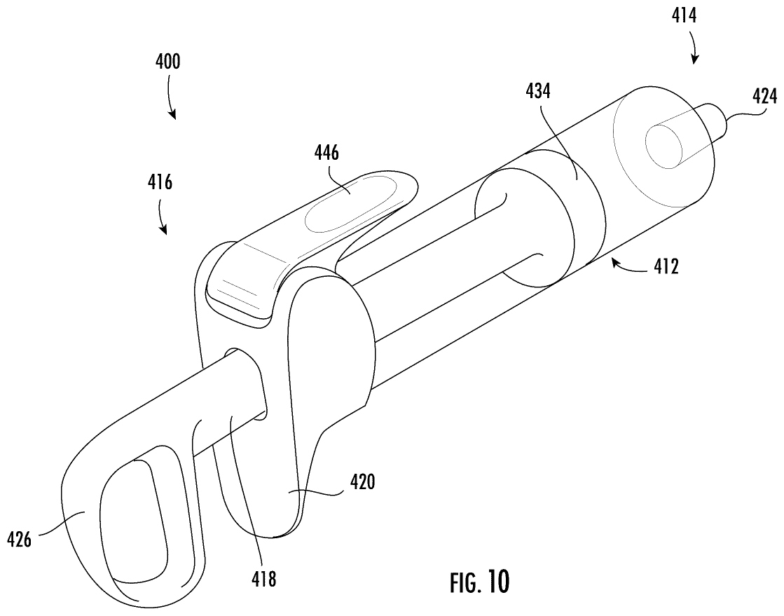

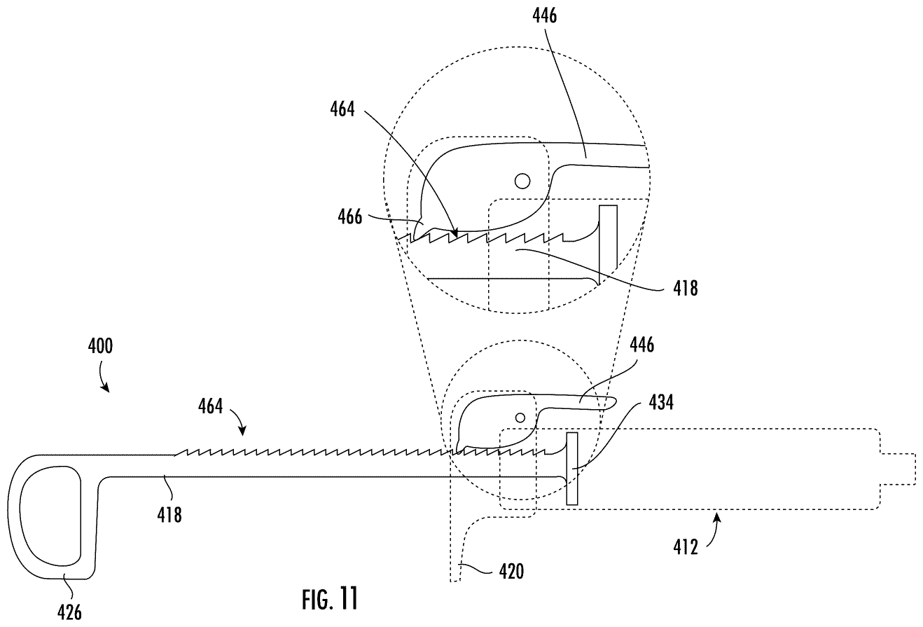

FIG. 10 is a perspective view of another example syringe 400. The syringe 400 may be similar in form and function to other syringes described herein. For example, the syringe 400 may include a barrel 412 having a lumen (e.g., inner chamber, cavity, etc.) within which a plunger 418 (e.g., piston) may be positioned. The barrel 412 of the syringe 400 may include a distal end region 414 and a proximal end region 416. In some instances, the proximal end region 416 of the barrel 412 may include one or more proximal finger flanges 420 (e.g., an ergonomic finger grip). It can be appreciated that a clinician may utilize the proximal finger flange 420 in conjunction with the plunger handle 426 to advance and/or retract the plunger 418 within the lumen of the barrel 412.

Additionally, FIG. 10 illustrates that the distal end region 414 of the barrel 412 may include one or more features designed to couple the barrel 412 of the syringe 400 to another medical device (e.g., an aspiration catheter). It can be appreciated that the distal end region 414 of the barrel 412 may include a coupling element 424 which permits the distal end region 414 of the barrel 412 to be rotated (e.g., screwed) and attached to a manifold of an aspiration catheter. For example, it can be appreciated that the coupling element 424 positioned along the distal end region 414 of the barrel 412 may include a standard luer fitting (e.g., luer lock) that can be coupled to a manifold of an aspiration catheter.

FIG. 10 further illustrates that the plunger 418 may include a stopper (e.g., gasket, etc.) 434 positioned along the distal end of the plunger 418. In some examples, the stopper 434 may include an O-ring which extends circumferentially around the outer surface of the distal end of the plunger 418. For example, the stopper 434 may include an O-ring which is nested in a circumferential groove formed in the outer surface of the plunger 418. It can be appreciated that the stopper 434 of the syringe 400 may be designed to seal against an inner surface of the of the barrel 412. Additionally, it can be further appreciated that the seal provided by the stopper 434 against the inner surface of the barrel 412 may permit the plunger 418 to generate a vacuum force when retracted in a distal-to-proximal direction relative to the barrel 412.

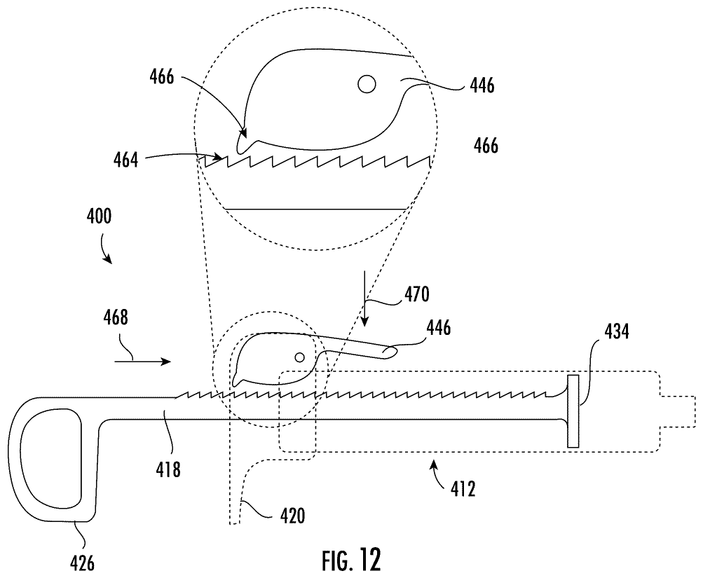

FIG. 10 further illustrates that the syringe 400 may further include an actuation lever 446 coupled to the barrel 412. As will be described in greater detail with respect to FIGS. 11-12, actuation of the actuation lever 446 may permit the distal advancement of the plunger 418 relative to the barrel 412.

FIG. 11 illustrates a side view of the syringe 400. For clarity, the barrel 412 of the syringe 400 is shown as a dashed outline. FIG. 11 illustrates that the plunger 418 may include a plurality of teeth 464 extending longitudinally along the outer surface of the plunger 418. Further, the detailed view of FIG. 11 illustrates that the actuation lever 446 may include a projection 466. The detailed view of FIG. 11 further illustrates the projection 466 engaged between a first tooth and a second tooth of the plurality of teeth 464. The syringe 400 may include a spring which is configured to maintain the actuation lever 446 in an engaged configuration until the actuation lever is pressed downward as illustrated in FIG. 12. It can be appreciated that the engagement of the projection 466 with the teeth 464 as illustrated in the detailed view of the FIG. 11 may prevent the plunger 418 from distal advancement of the plunger 418 within the lumen of the barrel 412. However, it can further be appreciated that engagement of the projection 466 with the teeth 464 as illustrated in the detailed view of the FIG. 11 may permit the plunger 418 to be proximally retracted relative to the barrel 412.

FIG. 12 illustrates a side view of the syringe 400. For clarity, the barrel 412 of the syringe 400 is shown as a dashed outline. FIG. 12 illustrates the actuation of the actuation lever 446. For example, the arrow 470 depicts the downward pressing of the actuation lever 446. Further, the detailed view of FIG. 12 illustrates that pressing the actuation lever 446 downward may permit the projection 466 to be released from engagement with the plurality of teeth 464. It can be appreciated that releasing the projection 466 from engagement with the teeth 464 as illustrated in the detailed view of the FIG. 11 may permit a clinician to proximally retract or distally advance the plunger 418 within the lumen of the barrel 412. The distal advancement of the plunger 418 (while the actuation lever 446 is being pressed downward) is depicted by the arrow 468 in FIG. 12. In some examples, movement of the plurality of teeth 464 may be used to determine how much volume is being dispensed from or into the barrel 412. In other words, each tooth of the plurality of the teeth 464 may correlate to a given volume of fluid in the barrel 412. Visual and/or audible indication (e.g., the mechanical sound generated by the plurality of teeth 464 passing the actuation lever 446) may aid a clinician in dispensing or extracting a specific or target volume of fluid from the barrel 412.

In one or more of the described embodiments, it can be appreciated that the plunger 418 of the syringe 400 can also be advanced or retracted without the need to actuate the lever.

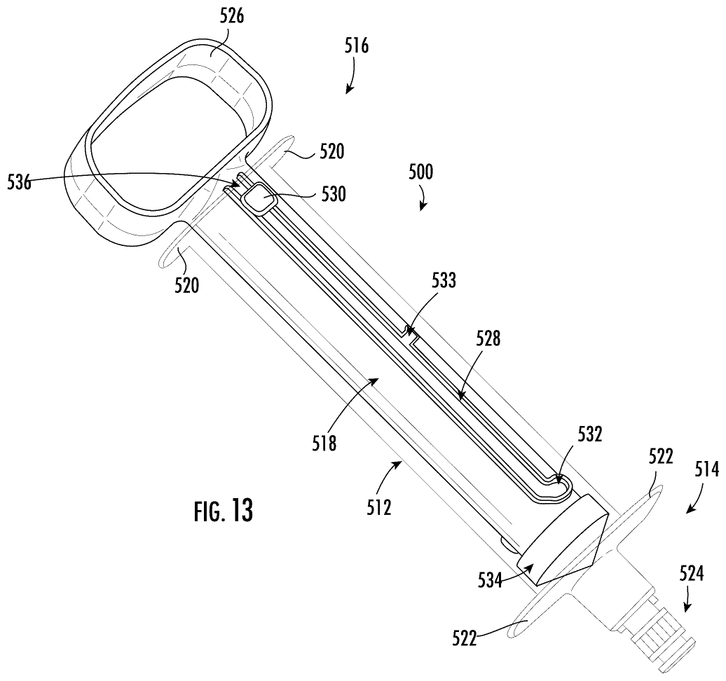

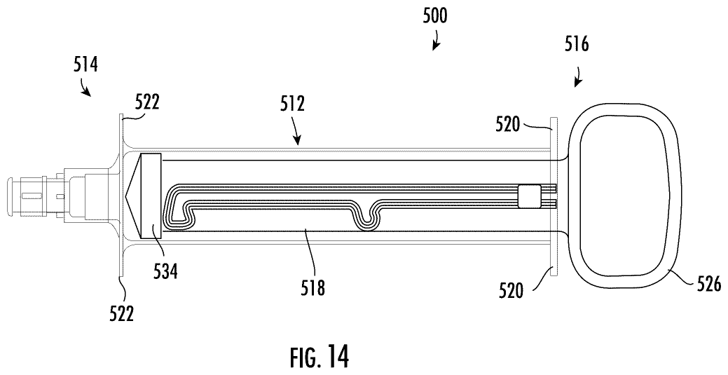

FIG. 13 is a perspective view of another example aspiration source 500 (e.g., syringe). It can be appreciated that the syringe 500 may include a barrel 512 having a lumen (e.g., inner chamber, cavity, etc.) within which a plunger 518 (e.g., piston) may be positioned. The barrel 512 of the syringe 500 may include a distal end region 514 and a proximal end region 516. In some instances, the proximal end region 516 of the barrel 512 may include one or more proximal finger flanges 520 (e.g., ergonomic finger grips) extending radially away from the outer surface of barrel 512. Additionally, the distal end region 514 of the barrel 512 may include one or more distal flanges 522 (e.g., ergonomic finger grips) extending radially away from the outer surface of barrel 512. Further, the proximal end of the plunger 518 may include a plunger handle 526 designed to permit a clinician to advance and/or retract the plunger 518 within the lumen of the barrel 512. It can be appreciated that a clinician may utilize the one or more proximal finger flanges 520 and or the distal flanges 522 in conjunction with the plunger handle 526 to advance and/or retract the plunger 518 within the lumen of the barrel 512. The distal flanges 522 may be gripped or wrapped by multiple fingers, a hand or first to provide additional support when advancing or retracting the plunger handle 526.

FIG. 13 illustrates the syringe 500 having the plunger 518 slidably and rotatably positioned within a lumen of the barrel 12. In some examples, the cross-sectional shape of the plunger 518 may be substantially cylindrical. However, in other examples, the cross-sectional shape of the plunger 518 may include other shapes, including ovular, triangular, square, rectangular, star-shaped, polygonal or other similar shapes. Further, in some examples, it can be appreciated that the plunger 518, the barrel 512 or both the plunger 518 and the barrel 512 may be constructed from a transparent material which permits visualization of contents within the lumen of the barrel 512. For example, the plunger 518, the barrel 512 or both the plunger 518 and the barrel 512 may be constructed from a polymeric, transparent material which may permit the visualization of a blood clot after being aspirated into the lumen of the barrel 512. An exemplary material for the plunger 518, the barrel 512, or both the plunger 518 and the barrel 512 is polycarbonate.

Additionally, FIG. 13 illustrates that the distal end region 514 of the barrel 512 may include one or more features designed to couple the barrel 512 of the syringe 500 to another medical device (e.g., an aspiration catheter). It can be appreciated that the distal end region 514 of the barrel 512 may include a coupling element 524 which permits the distal end region 514 of the barrel 512 to be rotated (e.g., screwed) and attached to a manifold of an aspiration catheter. For example, it can be appreciated that the coupling element 524 positioned along the distal end region 514 of the barrel 512 may include a standard luer fitting (e.g., luer lock) that can be coupled to a manifold of an aspiration catheter.

FIG. 13 further illustrates that the plunger 518 may include a stopper (e.g., gasket, etc.) 534 positioned along the distal end of the plunger 518. In some examples, the stopper 534 may include an O-ring which extends circumferentially around the outer surface of the distal end of the plunger 518. For example, the stopper 534 may include an O-ring which is nested in a circumferential groove formed in the outer surface of the plunger 518. It can be appreciated that the stopper 534 of the syringe 500 may be designed to seal against an inner surface of the of the barrel 12. Additionally, it can be further appreciated that the seal provided by the stopper 534 against the inner surface of the barrel 512 may permit the plunger 518 to generate a vacuum force when retracted in a distal-to-proximal direction relative to the barrel 512. Vacuum force is dependent upon several factors including the overall volume of the barrel 512.

FIG. 13 further illustrates that the plunger 518 may further include a channel 528 (e.g., motion channel, track, guide, groove, etc.) extending along the longitudinal length of the plunger 518. For example, FIG. 13 illustrates that the channel 528 may include a first end 536 positioned adjacent to the plunger handle 526 and a second end 532 positioned adjacent to the stopper 534, whereby the channel 528 extends longitudinally along the outer surface of the plunger 518 from the first end 536 to the second end 532. Additionally, FIG. 13 illustrates that the second end 532 of the channel may include a curved shape (e.g., “J-shape”), whereby the channel 528 transitions from extending along the longitudinal axis of the plunger 518 to extending along the circumference of the outer surface of the plunger 518. In other words, the channel 528 illustrated in FIG. 13 may extend along the outer surface of the plunger 518 from the first end 536 toward the second end 532, whereby the channel 528 bends approximately 90 degrees and extends along the outer circumference of the plunger 518.

FIG. 13 further illustrates that the channel 528 may include a locking channel 533 which extends along the circumference of the plunger 518. It can be appreciated from FIG. 13 that the locking channel 533 may be oriented substantially perpendicular to the channel 528. For example, the locking channel 533 may extend approximately 90 degrees relative to the channel 528. Further, FIG. 13 illustrates that the locking channel 533 may be spaced away from both the first end 536 and the second end 532 of the channel 528. In some examples, the locking channel 533 may be spaced approximately midway between the first end 536 and the second end 532 of the channel 528. However, it can be appreciated that the locking channel 533 may be positioned at any location along the plunger 518. It can be further appreciated that the position of the locking channel 533 may correspond to a given volume of fluid and/or vacuum generated when the plunger 518 is proximally retracted.

FIG. 13 further illustrates that the syringe 500 may further include a motion guide pin 530. It can be appreciated that at least a portion of the pin 530 may be disposed along the outer surface of the barrel 512 and that another portion of the pin 530 may extend through the wall of the barrel 512 and engage (e.g., nest within, project into, extend into, etc.) the channel 528. In other words, the pin 530 may include an attachment region which is attached to the outer surface of the barrel 512 and projection portion which extends through the wall of the barrel 512 and engages and/or projects into the channel 528.

FIGS. 14-16 illustrate the proximal retraction of the plunger 518 relative to the barrel 512 of the syringe 500. FIG. 14 illustrates the plunger 518 fully advanced in a distal direction within the barrel 512 whereby the plunger 518 has filled the entire space of the lumen of the barrel 518. In this configuration, a distally-facing surface of the stopper 534 may contact a proximal facing inner surface of the barrel 512. Further, it can be appreciated from FIG. 14 that the plunger handle 526 is positioned such that it is adjacent to the proximal finger flanges 520. It can be further appreciated that FIG. 14 may represent a pre-retraction configuration in which a clinician may position the plunger 518 to generate a maximum aspiration force (e.g., a maximum suction force) because the plunger 518 has extended a maximum distance into the barrel 512.

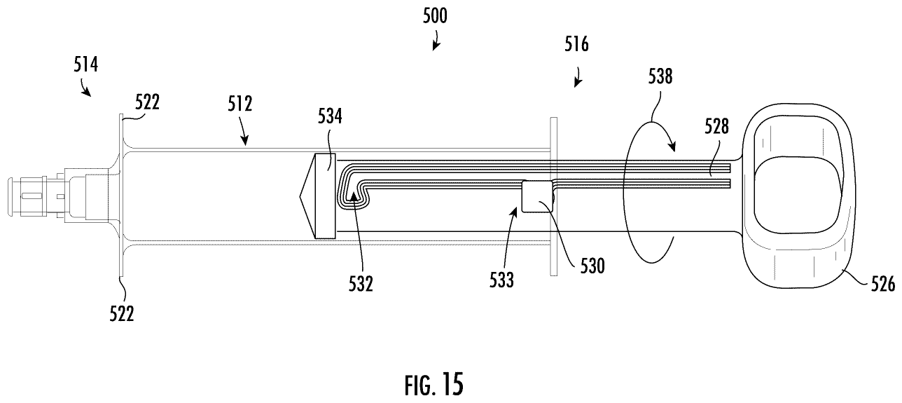

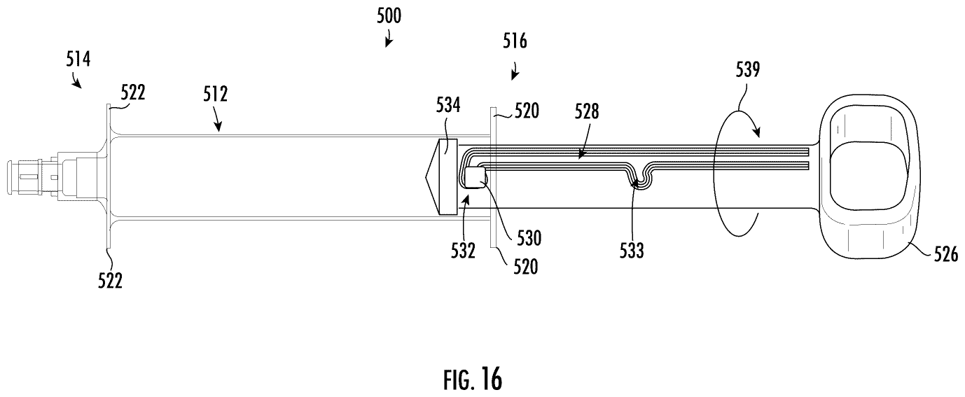

FIG. 15 illustrates that the syringe 500 shown in FIG. 13 after the plunger 518 has been retracted to a position in which the guide pin 530 engages the locking channel 533. It can be appreciated that to retract the plunger 518, a clinician may utilize the one or more of the proximal finger flanges 520 and or the distal flanges 522 in conjunction with the plunger handle 526 to retract the plunger 518 within the lumen of the barrel 512. Further, FIG. 15 illustrates that during the retraction of the plunger 518, the pin 530 may force the plunger 518 to be retracted along a longitudinal retraction path defined by the channel 528 and/or a locking channel 533. In other words, because the pin 530 is fixedly attached to the barrel 512, the pin 530 will maintain the plunger 518 on a retraction path defined by the orientation and shape of the channel 528 and/or a locking channel 533 relative to the barrel 512. Accordingly, FIG. 15 illustrates that, if desired, the plunger 518 may be retracted along the entire length of the channel 528. In other words, a clinician may retract the plunger 518 along the entire length of the channel 528 without rotating the plunger 518 such that the pin 530 engages the locking channels 533.

Additionally, it can be appreciated that during retraction of the plunger 518, a clinician may rotate the plunger 518 in a clockwise direction (as viewed from an end view of the handle 526) to engage the pin 530 within the locking channel 533 (rotation of the plunger to engage the pin 530 into the locking channel 533 is depicted by the arrow 538). For example, FIG. 15 illustrates the plunger 518 having been rotated such that the pin 530 is engaged with the locking channel 533. It can be appreciated that engagement of the plunger 518 in the locking channel 533 may maintain the plunger 518 in a retracted position whereby the plunger 518 will not advance in a proximal-to-distal direction due to the negative pressure being imparted on the plunger 518 via the retraction of the plunger 518. It can be further appreciated that to release the plunger 518 from the locking channel 533 and permit it to distally advance, a clinician may initiate rotation of the plunger 518 in a counter-clockwise direction (as viewed from an end view of the handle 526) thereby allowing the plunger 518 to advance along the channel 528 which is aligned with the longitudinal axis of the barrel 512.

As discussed herein, FIG. 16 further illustrates that during the retraction of the plunger 518, the engagement of the pin 530 within the channel 528 forces the plunger 518 to be retracted along a longitudinal retraction path which mirrors the orientation of the channel 528 relative to the barrel 512. Accordingly, FIG. 16 illustrates that the plunger 518 may be retracted along a retraction pathway which is void of any rotation relative to the barrel 512 up until the second end 532 of the channel 528 reaches the pin 530 (e.g., in some examples, the plunger 518 may bypass the locking channel 533), whereby continued engagement of the pin 530 within the channel 528 directs the plunger 518 to rotate (depicted by the arrow 539) in a clockwise direction (as viewed from and end view of the handle 526) whereby the pin 530 engages the second end 532 of the channel 528. It can be appreciated that engagement of the pin 530 within the second end 532 of the channel 28 may maintain the plunger 518 in a retracted position whereby the plunger 518 will not distally advance due to the negative pressure being imparted on the plunger 518 via the retraction of the plunger 518. It can be further appreciated that to release the plunger 518 and permit it to distally advance, a clinician may initiate rotation of the plunger 518 in a counter-clockwise direction (as viewed from an end view of the handle 526) thereby allowing the plunger 518 to advance along the portion of the channel 528 which is aligned with the longitudinal axis of the barrel 512. It can further be appreciated that, in some examples, a clinician may retract the plunger 518 in a stepwise manner, whereby the plunger 518 is first retracted to a position in which the pin 530 engages the locking channel 533, following by continued retraction in which the pin 530 engages the second end 532 of the channel 528.

Additionally, it can be appreciated that the syringe 500 (or any of the syringes disclosed herein) may be attached to an aspiration catheter. Further, the aspiration catheter may include a clamp, valve, or stopcock which may be utilized to control flow through the catheter. It can be further appreciated that, at the clinician's discretion, the clamp, valve, or stopcock may be opened whereby a vacuum pressure generated by the syringe 500 may create a vacuum force (e.g., suction) through the aspiration catheter which is attached to the syringe 500. It can be appreciated that the vacuum force generated through the aspiration catheter may aspirate blood clots or thrombus material located adjacent the distal end aspiration catheter.

It can be appreciated that any of the syringes disclosed herein may be utilized with a catheter aspiration system to remove blood clots from a blood vessel. For example, a catheter aspiration system including any of the syringes described herein may be utilized to generate suction forces for aspirating clot material from within a blood vessel. It can be appreciated that generating maximum suction forces to aspirate clot material may require air to be purged from an aspiration catheter coupled to any of the syringes described herein.

As herein, in some instances the clinician may utilize a clamp to control initiation of the vacuum force generated by any of the syringes disclosed herein. It can be further appreciated that, at the clinician's discretion, the clamp may be opened whereby a vacuum pressure generated by a syringe may create a vacuum force (e.g., suction) through the aspiration catheter which is attached to the syringe. It can be appreciated that the vacuum force generated through the aspiration catheter may aspirate blood clots or thrombus material located adjacent the distal end aspiration catheter.

The materials that can be used for the various components of the medical device 10 and the various other medical devices disclosed herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 and the various other medical devices disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 and the various other medical devices disclosed herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 and the various other medical devices disclosed herein to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A syringe, comprising:
a barrel having a distal end region, a proximal end region and an inner surface defining a lumen extending therein;
a plunger having a proximal end region, a distal end region and a channel, wherein the channel includes a proximal end region and a distal end region; and
an engagement member coupled to the barrel;
wherein the plunger is configured to be positioned within the lumen of the barrel;
wherein a portion of the engagement member extends into the channel of the plunger when the plunger is positioned within the lumen of the barrel.

2. The syringe of claim 1, wherein the distal end region of the barrel is configured to couple to a manifold of a catheter system.

3. The syringe of claim 1, wherein the channel extends along an outer surface of the plunger.

4. The syringe of claim 3, wherein the proximal end region of the channel is positioned along the proximal end region of the plunger, wherein the distal end region of the channel is positioned along the distal end region of the plunger, and wherein the channel extends from the proximal end region of the plunger to the distal end region of the plunger.

5. The syringe of claim 4, wherein a portion of the channel extending between the proximal end region of the plunger and the distal end region of the plunger is substantially parallel to the longitudinal axis of the plunger.

6. The syringe of claim 5, wherein the distal end region of the channel includes a curved shape.

7. The syringe of claim 6, wherein engagement of the engagement member within the curved shape of the channel is configured to prevent translation and rotation of the plunger within the barrel.

8. The syringe of claim 1, wherein the engagement member is positioned along the proximal end region of the barrel.

9. The syringe of claim 8, wherein the engagement member includes a guide pin, and wherein a portion of the guide pin extends through a wall of the barrel.

10. The syringe of claim 8, wherein the engagement member includes a spring.

11. The syringe of claim 10, wherein the syringe further comprises an actuation member configured to translate relative to the plunger, and wherein translation of the actuation member disengages the spring from the channel.

12. The syringe of claim 1, wherein the proximal end region of the barrel further includes a proximal flange, and wherein the distal end region of the barrel includes a distal flange.

13. The syringe of claim 1, wherein the channel includes a first portion extending along a longitudinal axis of the plunger and a second portion extending at an angle relative to the first portion.

14. The syringe of claim 1, wherein the distal end region of the plunger further includes a stopper, and where the stopper is configured to seal against the inner surface of the barrel.

15. A syringe, comprising:
a barrel having a distal end region, a proximal end region and an inner surface defining a lumen extending therein;
a plunger having a proximal end region, a distal end region, a handle and a first detent;
an actuation member coupled to the plunger; and
a spring coupled to the barrel, wherein the spring includes an engagement portion configured to engage the first detent;
wherein the plunger is configured to be positioned within the lumen of the barrel;
wherein engagement of the engagement portion of the spring within the first detent prevents translation of the plunger within the barrel.

16. The syringe of claim 15, further comprising a second detent, and wherein the first detent is spaced away from the second detent.

17. The syringe of claim 16, wherein the plunger further includes a channel, and wherein the actuation member is disposed within at least a portion of the channel.

18. The syringe of claim 17, wherein the actuation member is configured to translate relative to the plunger, and wherein translation of the actuation member disengages the spring from the first detent or the second detent.

19. The syringe of claim 18, wherein a proximal end of the actuation member extends through an aperture of the handle.

20. A syringe, comprising:
a barrel having a distal end region, a proximal end region and an inner surface defining a lumen extending therein;
a plunger having an outer surface and a plurality of teeth extending along the outer surface; and
an actuation lever coupled to the barrel, wherein the actuation lever includes a projection configured to engage a first tooth and a second tooth of the plurality of teeth;
wherein the plunger is configured to be positioned within the lumen of the barrel;
wherein engagement of the projection with the plurality of teeth prevents the plunger from translating distally relative to the barrel.
